(19) United States
(12) Patent Application Publication
(10) Pub. No.: US 2025/0282738 A1
(43) Pub. Date: Sep. 11, 2025

(54) Method for Improving Hydrogen Storage Performance of Covalent Organic Framework Compound, and Application Thereof for Hydrogen Storage

(71) Applicants: TSINGHUA UNIVERSITY, Beijing (CN); BEIJING VFORTUNE NEW ENERGY POWER TECHNOLOGY DEVELOPMENT CO., LTD., Beijing (CN)

(72) Inventors: Hong XU, Beijing (CN); Xiangming HE, Beijing (CN); Jia CHEN, Beijing (CN)

(21) Appl. No.: 19/218,427

(22) Filed: May 26, 2025

Related U.S. Application Data
(63) Continuation of application No. PCT/CN2024/071944, filed on Jan. 12, 2024.

(30) Foreign Application Priority Data
Jan. 12, 2023 (CN) 202310070508.8

(51) Int. Cl. C07D 251/56; C01B 3/00; C07C 211/50; C07C 211/54

(52) U.S. Cl. CPC C07D 251/56; C01B 3/0015; C07C 211/50; C07C 211/54

(57) ABSTRACT

The disclosure provides a method for improving hydrogen storage performance of a covalent organic framework compound, including: enabling an aromatic polyamino monomer and an aromatic polyaldehyde monomer to be subjected to dehydration and polycondensation to form the covalent organic framework compound, where the aromatic polyamino monomer and/or the aromatic polyaldehyde monomer contains at least one fluorinated aromatic ring, at least one hydrogen atom on the fluorinated aromatic ring is substituted with fluorine, hydrogen atoms not substituted with fluorine exist on the fluorinated aromatic ring, and the covalent organic framework compound has a two-dimensional or three-dimensional structure.

### TECHNICAL FIELD

The disclosure relates to the technical field of hydrogen storage materials, in particular to, a method for improving hydrogen storage performance of a covalent organic framework compound, and an application of a fluorinated covalent organic framework compound as a hydrogen storage medium.

### BACKGROUND

With the increasing demand and use of energy sources by humans, non-renewable energy sources such as fossil fuels (coal, petroleum and natural gas) will become increasingly depleted. The large-scale development and utilization of renewable energy sources become an important component of energy strategies in countries around the world. Hydrogen energy has the characteristics of abundant sources, environmental friendliness, renewability, high energy density and the like, and thus is the most ideal energy source in the future. One of the biggest technological obstacles to the use of hydrogen energy as a fuel is the storage problem thereof. Hydrogen is in a gaseous state at normal temperature and pressure, and the density of hydrogen is only 1/14 of that of air. A vehicle traveling 482.7 km (300 miles) consumes approximately 5-13 kg of hydrogen, while at normal temperature and pressure, 5 kg of hydrogen occupies up to 56 m³ of space. Obviously, the application of hydrogen-powered vehicles requires more practical and feasible hydrogen storage methods.

At present, many hydrogen storage methods have been proposed, and the most common methods include a compressed gas storage method and a liquid hydrogen storage method. Although the methods are easy to implement and have mature technologies, the methods all have respective defects that are difficult to overcome. For example, storing and transporting high pressure gaseous hydrogen in a steel cylinder has a certain risk. Hydrogen can dissolve and penetrate into a steel wall under high pressure, thereby causing the phenomenon of hydrogen embrittlement of the storage steel cylinder. This brings a huge potential safety hazard for long-term hydrogen storage. Moreover, the hydrogen storage capacity is relatively small, and the cost is high. Liquid hydrogen has a higher density than gaseous hydrogen, but the storage temperature for liquefied hydrogen is −252.8° C., which means that a large amount of energy and excellent insulation devices are required for storage. Therefore, requirements for devices are relatively high, and the cost is high. Therefore, it is extremely important to search for new hydrogen storage materials and storage and transportation methods. In the exploration of materials, porous materials become a recent hot topic.

Adsorption is a phenomenon of partial retention of gas and solid after contact, and may be divided into two categories: chemisorption and physisorption according to the difference in adsorption force, adsorption heat, adsorption rate, selectivity, adsorption temperature, pressure and the like. A main chemical hydrogen storage material is a metal hydride. The chemisorption is usually related to activation energy, which means that molecules attracted to the surface must first pass through an energy barrier and tightly bind to the surface to complete adsorption. Therefore, most chemisorption processes have relatively high reaction activation energy, desorption processes are slow, and some metal hydrides are even non-renewable. The physisorption hydrogen storage is a process of accumulating gas molecules on the surface of a material without undergoing chemical reactions with the material by virtue of the intermolecular interactions between the gas and the material to adsorb and store the gas. Due to the weak interaction force between hydrogen molecules and surfaces of adsorbent pores, the physisorption hydrogen storage has faster adsorption and desorption kinetics. Common working environments include relatively low temperature and relatively high pressure. Main physical hydrogen storage materials include porous materials such as zeolite, activated carbon, carbon nanotubes, and metal organic frameworks.

Metal-organic framework (MOF) materials and covalent organic framework (COF) materials are both rapidly developing crystalline porous materials in recent years. The MOF is mainly composed of nitrogen and oxygen porous organic ligands of aromatic acids or bases, hybridizing with inorganic metal centers through coordination bonds to form a three-dimensional network structure crystal. Therefore, the MOF is also known as a porous coordination polymer (PCP). Since the pore structure of the MOF is similar to that of zeolite but the skeleton has flexibility, the MOF is also known as “soft zeolite”. The first generation of MOF material was synthesized in the mid-1990s, and the pore structure of the MOF material requires the support of guest molecules. If the guest molecules are removed, the skeleton will collapse, resulting in an unstable pore structure. Subsequently, researchers began assembling anionic, cationic, and neutral ligands into coordination polymers to synthesize a new generation of MOF material. The organic ligand of this type of MOF material is mainly a carboxyl-containing organic anionic ligand, and sometimes is also mixed with a nitrogen-containing heterocyclic organic neutral ligand. This generation of MOF material makes up for the defects of the previous generation of MOF material. When guest molecules are introduced or removed or a certain external stimulus (such as pressure) is applied, the skeleton structure of the material will undergo some changes, but will not collapse. The covalent organic framework material is a type of new framework structure material synthesized in recent years, and may have one-dimensional, two-dimensional and three-dimensional crystal structures. The framework of this type of material only contains organic structural units which are connected through very strong covalent bonds (such as C—C, C—O, and B—O), where COF-6, COF-8, and COF-10 materials have a layered two-dimensional structure similar to graphite. In addition, COF-102, COF-105, and COF-108 materials are materials having three-dimensional structures formed by introducing triangular and tetrahedral nodes. This type of material has the advantages of relatively high porosity and specific surface area, excellent thermal stability, and easy functionalization. Compared with the MOFs material, the COF material has a lower crystal density so that the COF material is expected to be more effectively applied in gas storage. Furthermore, covalent bonds connecting COF building units are more stable than coordination bonds of the MOF, so that the material has higher stability and potential for further modification.

At present, for physical hydrogen storage materials, MOF/COF materials mainly improve the hydrogen storage performance of materials from the perspective of increasing the specific surface area and pore volume thereof.

For example, the MOF-5 is a typical example among many MOF compounds, and a framework [Zn₄O(bdc)₃] thereof is a three-dimensional network with a pcu topology formed by the interconnection of Zn₄O(—COO)₆ units and terephthalic acid radical bdc²⁻. The patent document WO2005003622A1 discloses a hydrogen storage container added with an MOF-5 material. Under the pressure of 3 bar, the hydrogen storage weight of the container added with the MOF-5 is increased by 1.46 times compared to that of the container without the MOF-5.

However, there is a contradiction between high specific surface area and material stability in physical hydrogen storage materials. MOF/COF materials with high specific surface areas are often prepared by highly reversible reactions. This also means that they have a stronger tendency to decompose (poor chemical stability). Secondly, for MOF/COF materials with high specific surface areas, pore collapse is also an unavoidable problem. For example, the MOF with a BET specific surface area exceeding 5000 m²/g often has relatively complex activation processes, and methods such as supercritical CO2 are required. It can be seen that a too high specific surface area causes problems in both chemical stability and pore structure collapse. In addition, even the current 7200 MOF material cannot meet the hydrogen storage density requirements set by the United States Department of Energy for hydrogen storage systems. Therefore, finding a way other than increasing the BET specific surface area is a direction worth exploring for MOF/COF type physical hydrogen storage materials.

Due to the presence of aromatic ring structures in COF materials (requirements for rigid structural elements), these building units are difficult to have functionality, and functionalization often requires further pore modification. For hydrogen storage properties, the adsorption heat of the aromatic ring structure is usually very high (˜4 kJ/mol), so that it is difficult to provide effective adsorption sites. An effective method is needed to improve the adsorption heat of adsorption sites so as to improve the hydrogen storage performance of materials.

### SUMMARY

As mentioned above, combining the properties of physical hydrogen storage materials and chemical hydrogen storage materials is the key to developing efficient hydrogen storage materials, and is also a major challenge in the field of hydrogen storage. After in-depth research, the inventor unexpectedly discovered that by the fluorination treatment of a specific site on an aromatic ring of a covalent organic framework structure, the hydrogen adsorption heat and the hydrogen storage capacity of a covalent organic framework (COF) compound can be significantly increased.

According to a first aspect of the disclosure, a method for improving hydrogen storage performance of a covalent organic framework compound is provided, including: enabling an aromatic polyamino monomer and an aromatic polyaldehyde monomer to be subjected to dehydration and polycondensation to form the covalent organic framework compound, where the aromatic polyamino monomer and/or the aromatic polyaldehyde monomer contains at least one fluorinated aromatic ring, at least one hydrogen atom on the fluorinated aromatic ring is substituted with fluorine, hydrogen atoms not substituted with fluorine exist on the fluorinated aromatic ring, and the covalent organic framework compound has a two-dimensional or three-dimensional structure.

According to a second aspect of the disclosure, an application of a fluorinated covalent organic framework compound as a hydrogen storage medium is provided, wherein the fluorinated covalent organic framework compound is formed by dehydration and polycondensation of an aromatic polyamino monomer and an aromatic polyaldehyde monomer, the aromatic polyamino monomer and/or the aromatic polyaldehyde monomer contains at least one fluorinated aromatic ring, at least one hydrogen atom on the fluorinated aromatic ring is substituted with fluorine, hydrogen atoms not substituted with fluorine exist on the fluorinated aromatic ring, and the fluorinated covalent organic framework compound has a two-dimensional or three-dimensional structure.

In some implementation solutions, the fluorinated aromatic ring is selected from a benzene ring, a naphthalene ring, an anthracene ring, a phenanthrene ring, or a pyrene ring.

In some implementation solutions, the fluorinated aromatic ring is a benzene ring.

In some implementation solutions, the aromatic polyaldehyde monomer contains one fluorinated aromatic ring, the fluorinated aromatic ring contains two para-aldehyde groups, and the aromatic polyamino monomer has one of the following structures:

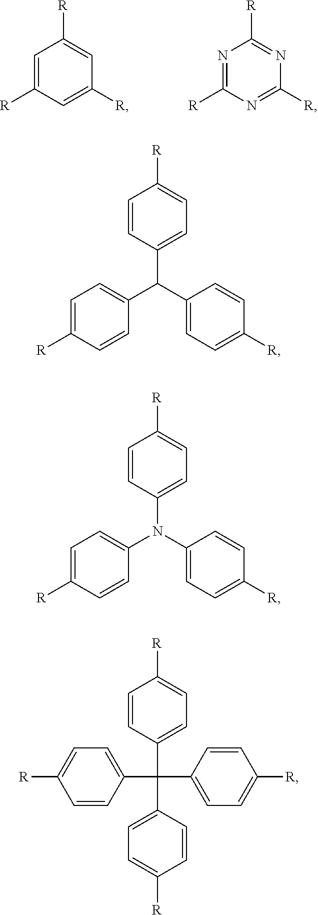

where R is amino or 4-aminophenyl.

In some implementation solutions, the aromatic polyamino monomer contains one fluorinated aromatic ring, the fluorinated aromatic ring contains two para-amino groups, and the aromatic polyaldehyde monomer has one of the following structures:

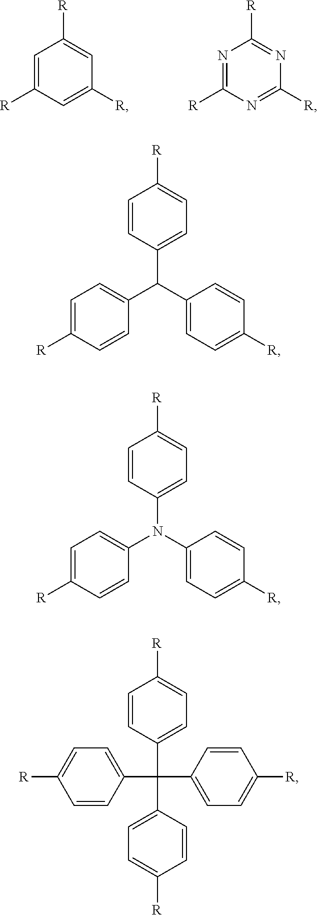

where R is aldehyde or 4-aldehyde phenyl.

In some implementation solutions, the aromatic polyamino monomer is 1,3,5-tris(4-aminophenyl)benzene or tetra(4-aminophenyl) methane.

In some implementation solutions, the aromatic polyaldehyde monomer is 2,5-difluoro-p-phthalaldehyde.

The disclosure has the following beneficial effects:
1. The modification treatment of a hydrogen storage material in the prior art is usually achieved by increasing the specific surface area of the material or doping the material to improve the hydrogen storage performance of the material, the operation is complex, and the cost is relatively high. The disclosure controls the adsorption induction force of a covalent organic framework compound through simple structural modification, which is favorable for solving the problem of low hydrogen storage capacity of an existing hydrogen storage material.
2. The low adsorption heat of a covalent organic framework compound in the prior art hinders the widespread promotion and application thereof as a hydrogen storage medium. The disclosure increases the hydrogen adsorption heat of a covalent organic framework compound by the fluorination treatment of a specific site on an aromatic ring of a COF, so as to increase the hydrogen storage capacity of the COF compound, which is favorable for promoting the practical application and development of the covalent organic framework in the field of hydrogen storage.
3. The method of the disclosure has universality and exhibits excellent implementation effects on covalent organic framework compounds of different structures and different dimensions.
4. Modification methods for hydrogen storage materials in the prior art are all limited to a milligram-level laboratory scale. The method of the disclosure can achieve amplified preparation, which is favorable for industrial applications.

### BRIEF DESCRIPTION OF FIGURES

The disclosure will be further described below with reference to accompanying drawings and implementations.

FIG. 1A shows a schematic diagram of a synthetic process of a two-dimensional covalent organic framework compound provided by the disclosure.

FIG. 1B shows a schematic diagram of a synthetic process of a three-dimensional covalent organic framework compound provided by the disclosure.

FIG. 2A to FIG. 2G respectively show X-ray diffraction (XRD) diagrams of covalent organic framework compounds provided by Preparation Example 1 to Preparation Example 7 of the disclosure.

FIG. 3A shows a nitrogen adsorption-desorption isotherm of a two-dimensional covalent organic framework compound provided by Preparation Example 1 of the disclosure.

FIG. 3B shows a nitrogen adsorption-desorption isotherm of a three-dimensional covalent organic framework compound provided by Preparation Example 2 of the disclosure.

FIG. 4A shows a hydrogen adsorption-desorption isotherm of a two-dimensional covalent organic framework compound of the disclosure.

FIG. 4B shows a hydrogen adsorption-desorption isotherm of a three-dimensional covalent organic framework compound of the disclosure.

FIG. 4C shows a hydrogen adsorption-desorption isotherm of a two-dimensional covalent organic framework compound provided by Preparation Example 4 of the disclosure.

FIG. 5A and FIG. 5B respectively show curve diagrams of the hydrogen adsorption capacity of two-dimensional and three-dimensional covalent organic framework compounds relative to pressure (bar).

### DETAILED DESCRIPTION

As used herein, the terms covalent organic framework, covalent organic skeleton and COF may be used interchangeably.

In order to make the technical solutions and advantages of the disclosure clearer and easier to understand, the disclosure is clearly and completely described below through examples with reference to accompanying drawings. It is to be noted that without conflicting with each other, various implementations or various technical features described below may be arbitrarily combined to form new implementations.

It is also to be noted that in the disclosure, words such as “exemplary” or “for example” are used to indicate examples, illustrations, or descriptions. Any implementation or design solution described as “exemplary” or “for example” in the disclosure is not to be interpreted as more preferred or advantageous than other implementations or design solutions. To be specific, the use of the word such as “exemplary” or “for example” is intended to present related concepts in a specific manner.

In the disclosure, “at least one” refers to one or more, and “multiple” refers to two or more. “And/or” describes an association relationship between associated objects, and indicates that there may be three relationships. For example, A and/or B may indicate the situation that A exists alone, A and B exist simultaneously, or B exists alone, where A and B may be singular or plural. The phrase “at least one of the following items (pieces)” or a similar expression thereof refers to any combination of these items, including any combination of a single item (piece) or multiple items (pieces). For example, at least one item (piece) of a, b or c may indicate: a, b, c, a, and b, a, and c, b, and c, a, and b, and c, where a, b and c may be singular or plural. It is worth noting that “at least one item (piece)” may also be interpreted as “one item (piece) or multiple items (pieces)”.

In the disclosure, the term “fluorinated aromatic ring” refers to the substitution of H on an aromatic ring with F, and the substitution of H on a substituent connected to the aromatic ring with F does not belong to the “fluorinated aromatic ring” in the disclosure.

As described above, the disclosure provides a method for improving hydrogen storage performance of a covalent organic framework compound, including: an aromatic polyamino monomer and an aromatic polyaldehyde monomer are subjected to dehydration and polycondensation to form the covalent organic framework compound, where the aromatic polyamino monomer and/or the aromatic polyaldehyde monomer contains at least one fluorinated aromatic ring, at least one hydrogen atom on the fluorinated aromatic ring is substituted with fluorine, hydrogen atoms not substituted with fluorine exist on the fluorinated aromatic ring, and the covalent organic framework compound has a two-dimensional or three-dimensional structure.

According to the disclosure, the aromatic polyamino monomer is an aromatic compound containing two or more amino groups. For example, the aromatic polyamino monomer may be selected from substituted or unsubstituted p-phenylenediamine, substituted or unsubstituted triaminobenzene, and other substituted or unsubstituted polyamino aromatic compounds, substituted or unsubstituted diamino heterocyclic compounds, substituted or unsubstituted triamino heterocyclic compounds or substituted or unsubstituted polyamino heterocyclic compounds.

According to the disclosure, the aromatic polyaldehyde monomer is an aromatic compound containing two or more aldehyde groups. For example, the polyaldehyde monomer is selected from substituted or unsubstituted p-phthalaldehyde, substituted or unsubstituted 2-phenylbenzaldehyde, substituted or unsubstituted p-2-thiophenealdehyde, and other substituted or unsubstituted polyaldehyde aromatic compounds, substituted or unsubstituted dialdehyde heterocyclic compounds, substituted or unsubstituted trialdehyde heterocyclic compounds or substituted or unsubstituted polyaldehyde heterocyclic compounds.

According to a preferred implementation solution of the disclosure, the aromatic polyaldehyde monomer contains one fluorinated aromatic ring, the fluorinated aromatic ring contains two para-aldehyde groups, and the aromatic polyamino monomer has one of the following structures:

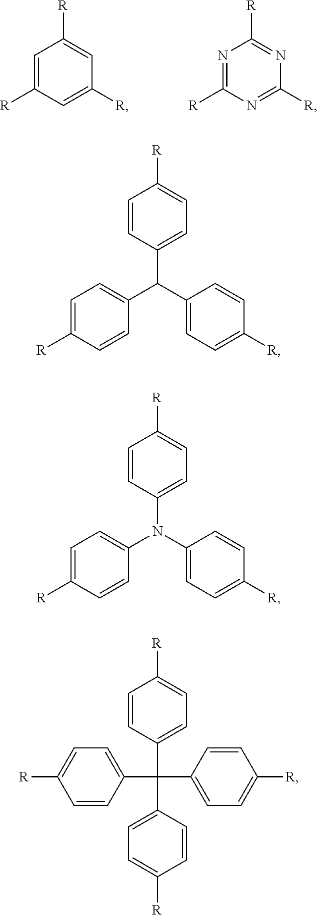

where R is amino or 4-aminophenyl.

In another preferred implementation solution, the aromatic polyamino monomer contains one fluorinated aromatic ring, the fluorinated aromatic ring contains two para-amino groups, and the aromatic polyaldehyde monomer has one of the following structures:

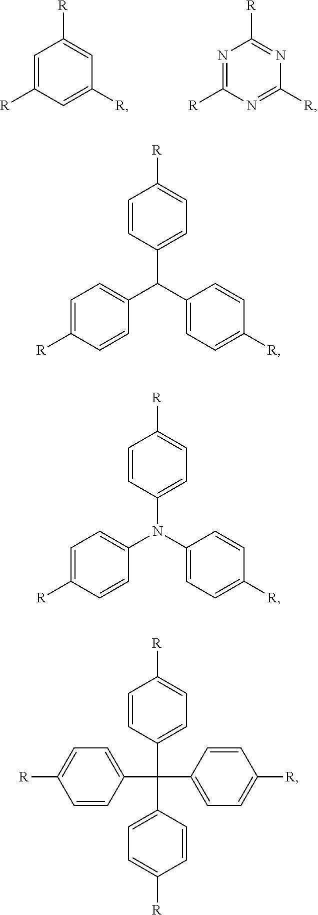

where R is aldehyde or 4-aldehyde phenyl.

According to the disclosure, the two-dimensional or three-dimensional structure refers to a structure with periodic repeated two-dimensional or three-dimensional crystal structural units.

The two-dimensional structure may be a two-dimensional sheet structure with horizontally arranged polygonal structural units having holes inside. The polygon may be, for example, a regular triangle or an irregular triangle, a quadrilateral, a pentagon, a hexagon, or a combination thereof, and has holes inside. Considering the stability of a chemical structure, the polygon may be, for example, a regular triangle, a regular quadrilateral, a regular pentagon, a regular hexagon, or a combination thereof, or for example, a regular pentagon, a regular hexagon, or a combination thereof, or for example, a regular hexagon.

The three-dimensional structure may be any type of three-dimensional crystal structure that exists in an overlapping stacked structure, staggered stacked structure, unidirectional stacked structure, or random stacked structure, and has pores inside.

According to the disclosure, the dehydration and polycondensation of the aromatic polyamino monomer and the aromatic polyaldehyde monomer to form the covalent organic framework compound may be any dehydration and polycondensation reactions well-known in the art. For example, the dehydration and polycondensation are carried out by taking a polyamino monomer and a polyaldehyde monomer as reactants to react in a mixed solvent under the catalysis of a catalyst. In general, the reaction is carried out at 100-150° C., preferably at 100-130° C., more preferably at 110° C., 112° C., 114° C., 116° C., 118° C. or 120° C., and most preferably at 120° C. The catalyst may be selected from any catalyst well-known in the art. For example, the catalyst may be selected from one or more of formic acid, acetic acid, p-toluenesulfonic acid, oxalic acid, lactic acid, hydrochloric acid, sulfuric acid, and pyrrolidine. Preferably, the catalyst is selected from acetic acid. The mixed solvent may be selected from any mixed solvent well-known in the art. For example, the mixed solvent may be any one of ethylene glycol+cyclohexane, mesitylene+dioxane, n-butanol+dioxane, o-dichlorobenzene+n-butanol, and mesitylene+n-butanol. In the mixed solvent, the volume ratio of the former to the latter is 9:1 to 1:9, for example, 5:1 to 1:5, and 3:1 to 1:3. Preferably, the volume ratio of two liquids is 1:1.

According to the disclosure, at least one hydrogen atom on the fluorinated aromatic ring is substituted with fluorine, and hydrogen atoms not substituted with fluorine exist on the fluorinated aromatic ring. Preferably, the fluorinated aromatic ring is a fluorinated benzene ring. More preferably, the fluorinated aromatic ring is a fluorinated benzene ring, the aromatic polyaldehyde monomer contains the fluorinated benzene ring, and the aromatic polyamino monomer does not contain the fluorinated benzene ring.

According to the disclosure, the fluorinated aromatic ring is connected with two para-aldehyde groups or two para-amino groups and has at least one hydrogen atom substituted with fluorine, and hydrogen atoms not substituted with fluorine exist on the fluorinated aromatic ring. Preferably, the fluorinated aromatic ring is connected with two para-aldehyde groups and has at least one hydrogen atom substituted with fluorine, and hydrogen atoms not substituted with fluorine exist on the fluorinated aromatic ring. More preferably, the fluorinated aromatic ring is connected with two para-aldehyde groups and has one, two, or three hydrogen atoms substituted with fluorine. Most preferably, the fluorinated aromatic ring is connected with two para-aldehyde groups, and the fluorinated aromatic ring is partially fluorinated.

The inventor unexpectedly discovered that compared with COF compounds containing perfluorinated aromatic rings and non-fluorinated COF compounds, COF compounds containing monofluoro-fluorinated aromatic rings, COF compounds containing difluoro-fluorinated aromatic rings, and COF compounds containing trifluoro-fluorinated aromatic rings have better hydrogen storage performance. In particular, by comparing COF compounds containing partially fluorinated aromatic rings with COF compounds containing perfluorinated aromatic rings, COF compounds containing partially fluorinated aromatic rings simultaneously have a much higher specific surface area and higher hydrogen storage capacity. In addition, in order to improve the hydrogen storage capacity, a fluorination site is to be located on an aromatic ring (such as a benzene ring), rather than on other substituents connected to the aromatic ring (such as the benzene ring).

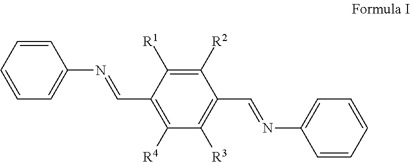

Formula I shows partial molecular structural units of a two-dimensional COF compound in a preparation example of the disclosure, where substituents R¹ to R⁴ on the benzene ring are independently selected from H, OCH₃, and F. The inventor conducted theoretical research and listed the dipole moment (molecular polarization index) under different substituent conditions in Table 1. From Table 3, it can be seen that the introduction of monofluoro substituents, partially fluorinated substituents, and trifluoro substituents improves the polarity of a substitution site on the benzene ring. The inventor discovered that introducing highly polar groups at specific sites of aromatic rings can regulate the environment in pores of the COF to increase the interaction with hydrogen molecules, which is an effective strategy for improving the adsorption heat of the material, thereby being favorable for improving the hydrogen storage performance of the COF material.

TABLE 1

| | | | | | Dipole moment |
|---|---|---|---|---|---|
| Number | R¹ | R² | R³ | R⁴ | (eV) |
| 1 | H | H | H | H | 0.37684808 |
| 2 | F | H | H | H | 0.39489081 |
| 3 | F | H | F | H | 0.41059401 |
| 4 | F | F | F | H | 0.40033184 |
| 5 | F | F | F | F | 0.38501040 |
| 6 | H | OCH₃ | H | OCH₃ | 0.38234260 |

The preferred conditions of the disclosure are further described in conjunction with examples and with reference to accompanying drawings. It is to be understood that the preferred examples described herein are only used to illustrate and explain the disclosure, but are not intended to limit the disclosure.

Raw materials or reagents used in the following preparation examples are all commercially available or self-made.

### Preparation Example 1

### Synthesis of Two-Dimensional Fluorinated Covalent Organic Framework Compound TPB-DFTP-COF

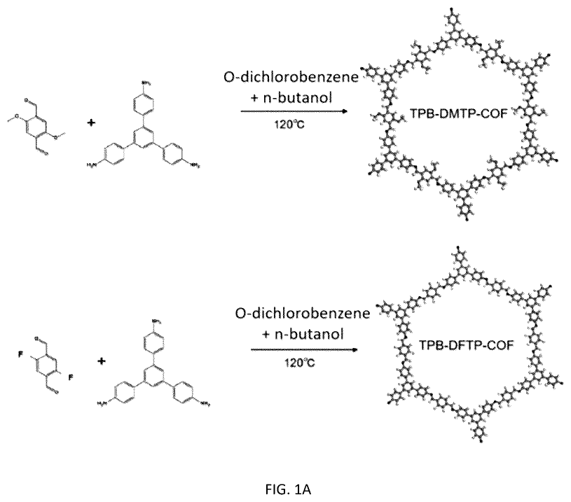

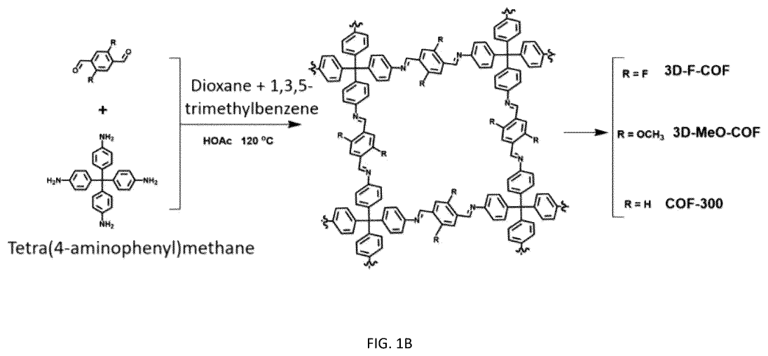

1,3,5-tris(4-aminophenyl)benzene (TPB) (0.1 mmol) and 2,5-difluoro-p-phthalaldehyde (DFTP) (0.15 mmol) were added to a mixed solvent of o-dichlorobenzene+n-butanol (o-DCB+n-BuOH, 1 mL, 1:1 volume ratio) and dissolved in the mixed solvent to obtain a mixture. Acetic acid (6 mol/L, 0.1 mL) was added to the mixture, the mixture was heated to 120° C. for thermal insulation reaction for 3 days, and the reaction product was filtered, washed and purified to obtain a fluorinated two-dimensional COF, named a TPB-DFTP-COF. The schematic diagram of the synthetic process refers to FIG. 1A.

### Preparation Example 2

### Synthesis of Three-Dimensional Fluorinated Covalent Organic Framework Compound 3D-F—COF

Tetra(4-aminophenyl) methane (0.1 mmol) and 2,5-difluoro-p-phthalaldehyde (0.2 mmol) were added to a mixed solvent of dioxane+1,3,5-trimethylbenzene (1 mL, 1:1 volume ratio) and dissolved in the mixed solvent to obtain a mixture. Acetic acid was added to the mixture, the mixture was heated to 120° C. for thermal insulation reaction for 3 days, and the reaction product was subjected to centrifugal separation and then subjected to Soxhlet extraction and purification with tetrahydrofuran to obtain a fluorinated three-dimensional COF, named a 3D-F—COF. The schematic diagram of the synthetic process refers to FIG. 1B.

### Preparation Example 3 (Comparison)

### Synthesis of Two-Dimensional Methoxylated Covalent Organic Framework Compound TPB-DMTP-COF

1,3,5-tris(4-aminophenyl)benzene (0.1 mmol) and 2,5-dimethoxy-p-phthalaldehyde (DMTP) (0.15 mmol) were added to a mixed solvent of o-dichlorobenzene+n-butanol (1 mL, 1:1 volume ratio) and dissolved in the mixed solvent to obtain a mixture. Acetic acid was added to the mixture, the mixture was heated to 120° C. for thermal insulation reaction for 3 days, and the reaction product was filtered, washed and purified to obtain a two-dimensional methoxylated COF, named a TPB-DMTP-COF. The schematic diagram of the synthetic process refers to FIG. 1A.

### Preparation Example 4 (Comparison)

### Synthesis of Two-Dimensional Perfluorinated Covalent Organic Framework Compound TPB-TFTP-COF

1,3,5-tris(4-aminophenyl)benzene (0.1 mmol) and 2,3,5,6-tetrafluoro-p-phthalaldehyde (0.15 mmol) were added to a mixed solvent of o-dichlorobenzene+n-butanol (1 mL, 1:1 volume ratio) and dissolved in the mixed solvent to obtain a mixture. Acetic acid was added to the mixture, the mixture was heated to 120° C. for thermal insulation reaction for 3 days, and the reaction product was filtered, washed and purified to obtain a two-dimensional perfluorinated COF, named a TPB-TFTP-COF.

### Preparation Example 5 (Comparison)

### Synthesis of Two-Dimensional Substituent-Free Covalent Organic Framework Compound

1,3,5-tris(4-aminophenyl)benzene (0.1 mmol) and p-phthalaldehyde (0.15 mmol) were added to a mixed solvent of o-dichlorobenzene+n-butanol (1 mL, 1:1 volume ratio) and dissolved in the mixed solvent to obtain a mixture. Acetic acid was added to the mixture, the mixture was heated to 120° C. for thermal insulation reaction for 3 days, and the reaction product was filtered, washed and purified to obtain a two-dimensional fluorine-free COF, named a 2D-F-free-COF.

### Preparation Example 6 (Comparison)

### Synthesis of Three-Dimensional Methoxylated Covalent Organic Framework Compound 3D-MeO—COF

Tetra(4-aminophenyl) methane (0.1 mmol) and 2,5-dimethoxy-p-phthalaldehyde (0.2 mmol) were added to a mixed solvent of dioxane+1,3,5-trimethylbenzene (1 mL, 1:1 volume ratio) and dissolved in the mixed solvent to obtain a mixture. Acetic acid was added to the mixture, the mixture was heated to 120° C. for thermal insulation reaction for 3 days, and the reaction product was subjected to centrifugal separation and then subjected to Soxhlet extraction and purification with tetrahydrofuran to obtain a fluorinated three-dimensional methoxylated COF, named a 3D-MeO—COF, referring to FIG. 1B.

### Preparation Example 7 (Comparison)

### Synthesis of Three-Dimensional Substituent-Free Covalent Organic Framework Compound COF-300

Tetra(4-aminophenyl) methane (0.1 mmol) and p-phthalaldehyde (0.2 mmol) were added to a mixed solvent of dioxane+1,3,5-trimethylbenzene (1 mL, 1:1 volume ratio) and dissolved in the mixed solvent to obtain a mixture. Acetic acid was added to the mixture, the mixture was heated to 120° C. for thermal insulation reaction for 3 days, and the reaction product was subjected to centrifugal separation and then subjected to Soxhlet extraction and purification with tetrahydrofuran to obtain a fluorinated three-dimensional COF, named a COF-300, referring to FIG. 1B.

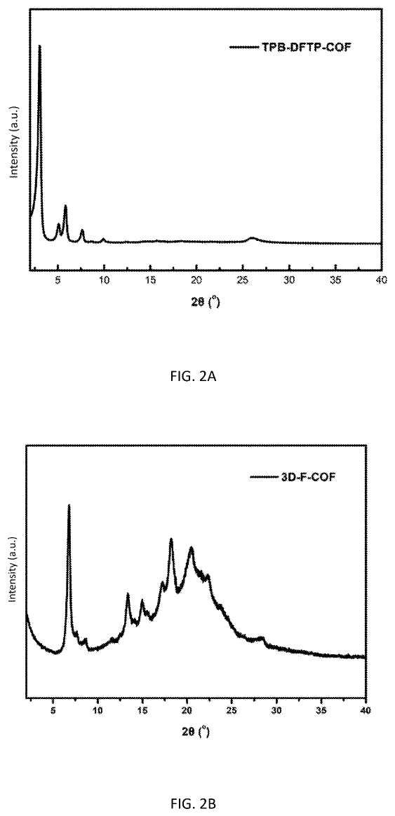

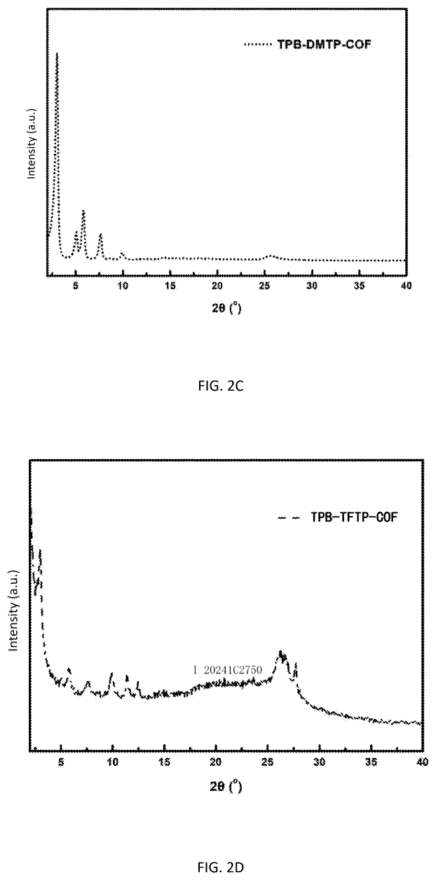

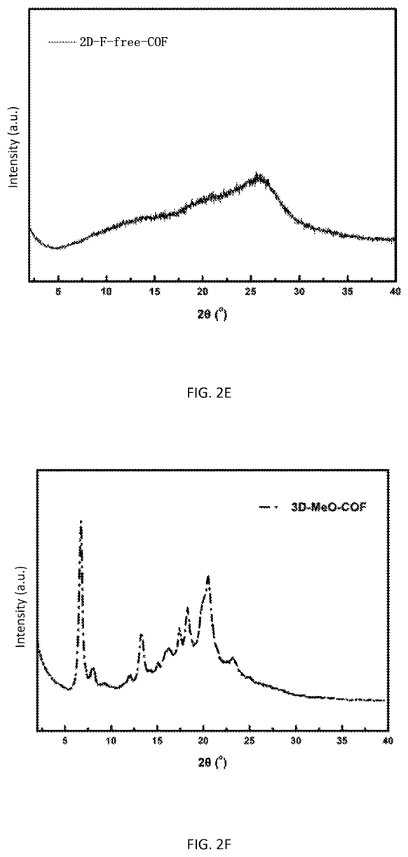

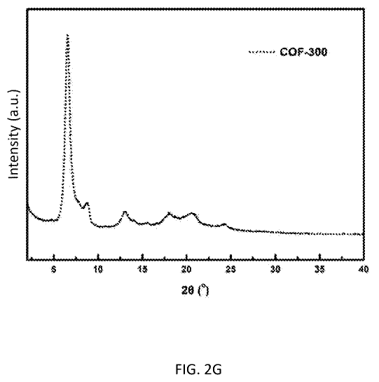

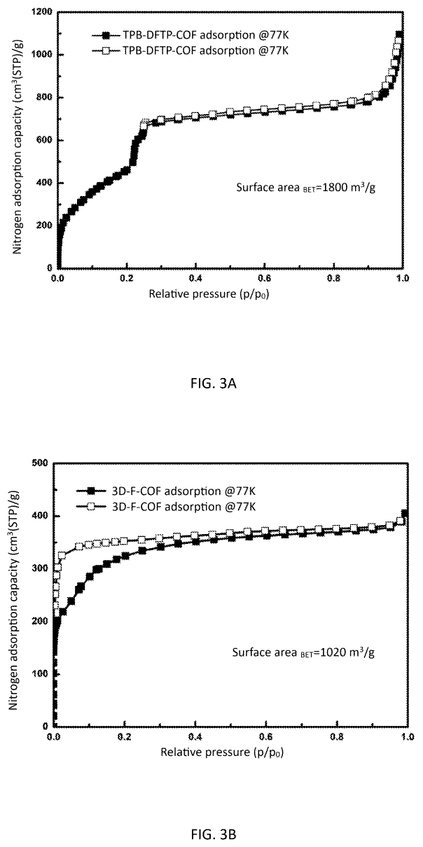

Table 2 lists the properties of the compounds obtained in Preparation Examples 1 to 7, and crystal structures thereof are analyzed by powder crystal X-ray diffraction (XRD). The obtained results refer to FIG. 2A to FIG. 2G.

TABLE 2

| Preparation Example | Compound | Property |
|---|---|---|
| 1 | TPB-DFTP-COF | Crystal powder |
| 2 | 3D-F-COF | Crystal powder |
| 3 (Comparison) | TPB-DMTP-COF | Crystal powder |
| 4 (Comparison) | TPB-TFTP-COF | Crystal powder |
| 5 (Comparison) | 2D-F-free-COF | Low-crystallinity powder |
| 6 (Comparison) | 3D-MeO-COF | Crystal powder |
| 7 (Comparison) | COF-300 | Crystal powder |

Referring to FIG. 2A and FIG. 2C, comparing powder crystal X-ray diffraction results of the TPB-DMTP-COF and the TPB-DFTP-COF, it indicates that compared with the two-dimensional covalent organic framework compound with methoxy groups, there is no significant difference in the crystal structure of the two-dimensional covalent organic framework compound with fluorine groups. Referring to FIG. 2B and FIG. 2F, comparing powder crystal X-ray diffraction results of the 3D-F—COF and the 3D-MeO—COF, it further indicates that the introduction of fluorine groups also has no significant effect on the crystal structure of the three-dimensional covalent organic framework compound.

FIG. 2(e) shows an X-ray diffraction pattern of the two-dimensional fluorine-free covalent organic framework compound obtained in Preparation Example 5, where there are no obvious crystal characteristic peaks, indicating poor crystallinity of the 2D-F-free-COF.

### Example 1

### (Measurement of BET Specific Surface Area and Measurement of Hydrogen Storage Capacity)

A gas adsorption instrument was used for measuring the BET specific surface area and hydrogen storage capacity of a portion of the obtained covalent organic framework compound.

The used normal pressure gas adsorption instrument was BELSORP-maxll manufactured by MicrotracBEL.

The used high pressure hydrogen adsorption instrument was an HPVA-100 high pressure volume analyzer manufactured by MICROMERITICS INSTRUMENT CORPORATION.

A method for measuring the BET specific surface area was as follows: an N₂ adsorption isotherm at 77 K was used for determination, and a BET (Brunauer-Emmett-Teller) equation was used for calculating the surface area of the material.

A method for measuring the hydrogen storage capacity at 77K-normal pressure was as follows: a normal pressure gas adsorption analyzer was used for obtaining an adsorption isotherm of hydrogen by a dynamic volumetric method.

A method for measuring the hydrogen storage capacity at 77K-high pressure was as follows: a high pressure gas adsorption analyzer was used for obtaining a high pressure adsorption isotherm of hydrogen by a static volumetric method.

The measured BET specific surface area, hydrogen storage capacity at 77K-normal pressure, and hydrogen storage capacity at 77K-high pressure (80 bar) were shown in Table 3.

TABLE 3

| | | | Hydrogen storage | |
|---|---|---|---|---|
| | | BET specific | capacity at 77 K- | Hydrogen storage |
| Preparation | | surface area | normal pressure | capacity at 77 K-80 |
| Example | Compound | (m³/g) | (wt %) | bar (wt %) |
| 1 | TPB-DFTP-COF | 1800 | 1.13 | 5.11 |
| 2 | 3D-F-COF | 1020 | 1.21 | 4.95 |
| 3 | TPB-DMTP-COF | 1880 | 0.84 | 3.52 |
| 4 | TPB-TFTP-COF | 677 | 0.38 | — |
| 5 | 2D-F-free-COF | — | — | — |
| 6 | 3D-MeO-COF | 1100 | 0.36 | 2.05 |
| 7 | COF-300 | 982 | 0.91 | 3.37 |

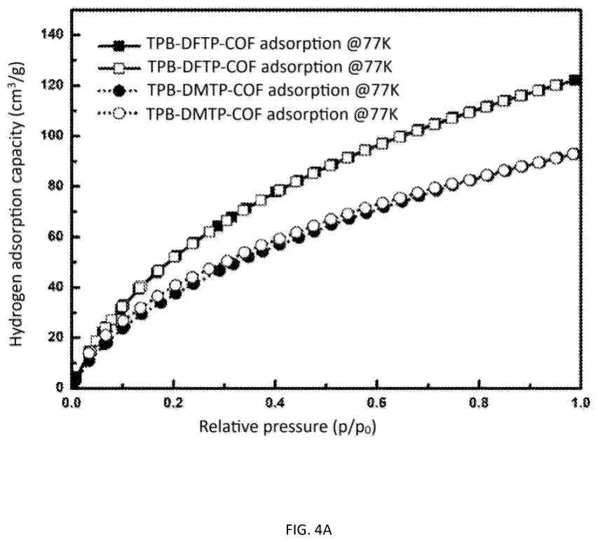

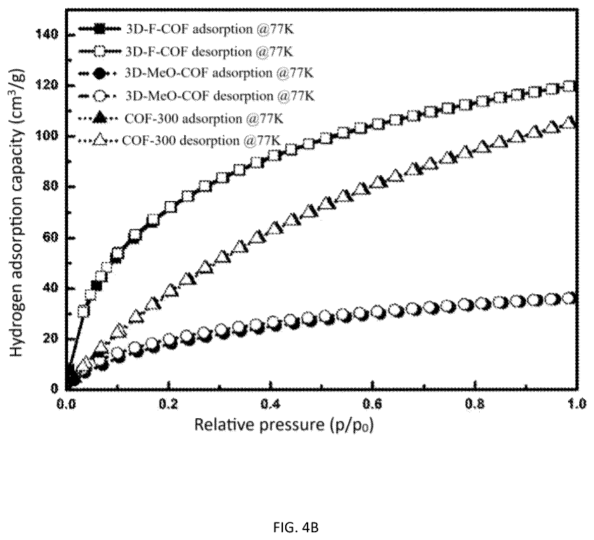

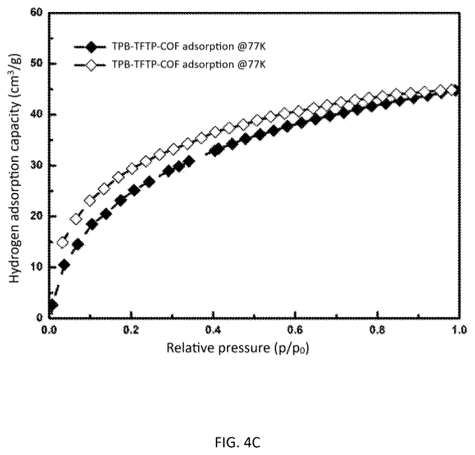

As shown in FIG. 4A to FIG. 4C, the inventor discovered that for both the two-dimensional COF compound and the three-dimensional COF compound, compared to the methoxylated COF and perfluorinated COF, the specific surface area of the partially fluorinated COF has no significant change, and the hydrogen adsorption capacity is significantly increased. It can be seen that the improvement in hydrogen adsorption performance of the COF compound obtained by fluorination treatment through a fluoride substitution method is not due to an increase in specific surface area, but rather due to the introduction of partially fluorinated substituents, which leads to an improvement in hydrogen adsorption performance. Therefore, the fluorination treatment introducing partially fluorinated substituents is a simple and widely applicable strategy to effectively improve the hydrogen storage performance of the COF compound.

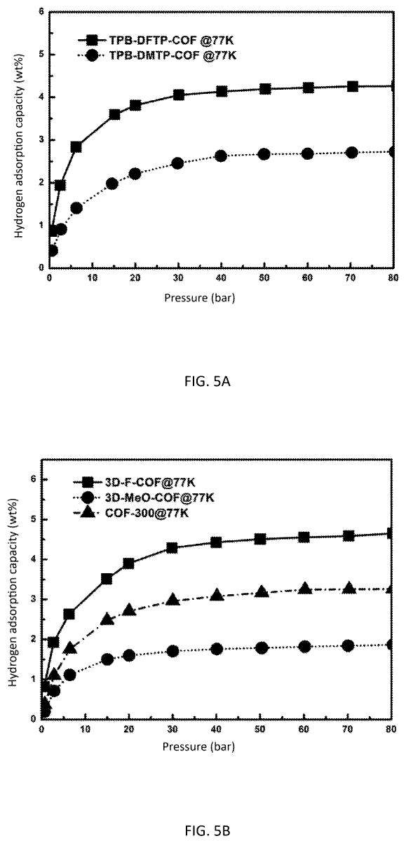

Referring to FIG. 5A and FIG. 5B, through the fluorination treatment method of the disclosure, the hydrogen storage capacities of both two-dimensional and three-dimensional covalent organic framework compounds are significantly increased under high pressure hydrogen storage conditions.

The inventor of the disclosure performs the simulated calculation of the adsorption energy of the material obtained by the method of the disclosure, and the obtained results are listed in Table 4 to evaluate the effect of the method of the disclosure on the hydrogen adsorption performance of the material. The simulated calculation is performed through a built-in module CASTEP of material calculation software Materials Studio (commercially available from Accelrys, USA). Simulated calculation results indicate that the fluorination treatment method introducing partially fluorinated substituents can significantly increase the adsorption energy.

TABLE 4

| | | Hydrogen adsorption | Adsorption energy |
|---|---|---|---|
| Preparation Example | Compound | site | kJ/mol |
| 1 | TPB-DFTP-COF | Imine bond | −6.07 |
| | | Fluoro | −4.18 |
| 2 | 3D-F-COF | Aldehyde benzene ring | −14.39 |
| | | Amino benzene ring | −11.50 |
| | | Imine bond | −10.16 |
| 3 (Comparison) | TPB-DMTP-COF | Imine bond | −4.52 |
| | | Methoxy | −2.59 |
| 6 (Comparison) | 3D-MeO-COF | Aldehyde benzene ring | −12.68 |
| | | Amino benzene ring | −10.27 |
| | | Imine bond | −9.94 |
| 7 (Comparison) | COF-300 | Aldehyde benzene ring | −12.21 |
| | | Amino benzene ring | −10.08 |
| | | Imine bond | −9.32 |

### INDUSTRIAL APPLICABILITY

The covalent organic framework compound obtained by the processing method of the disclosure can store hydrogen at a practical level, so that the utilization of hydrogen is easier. With the advent of the hydrogen society, the processing method will have more universal practical value.

What is claimed is:

1. A method for improving hydrogen storage performance of a covalent organic framework compound, comprising: enabling an aromatic polyamino monomer and an aromatic polyaldehyde monomer to be subjected to dehydration and polycondensation to form the covalent organic framework compound, wherein the aromatic polyamino monomer and/or the aromatic polyaldehyde monomer contains at least one fluorinated aromatic ring, at least one hydrogen atom on the fluorinated aromatic ring is substituted with fluorine, hydrogen atoms not substituted with fluorine exist on the fluorinated aromatic ring, and the covalent organic framework compound has a two-dimensional or three-dimensional structure.

2. The method according to claim 1, wherein the fluorinated aromatic ring is selected from a benzene ring, a naphthalene ring, an anthracene ring, a phenanthrene ring, or a pyrene ring.

3. The method according to claim 1, wherein the fluorinated aromatic ring is a benzene ring.

4. The method according to claim 1, wherein the aromatic polyaldehyde monomer contains one fluorinated aromatic ring, the fluorinated aromatic ring contains two para-aldehyde groups, and the aromatic polyamino monomer has one of the following structures: 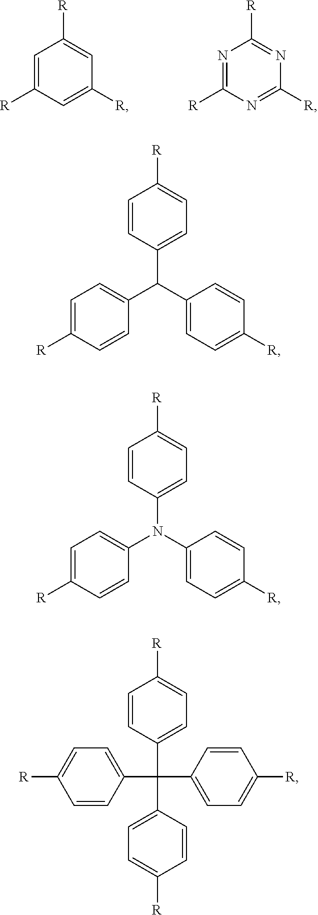
wherein R is amino or 4-aminophenyl.

5. The method according to claim 1, wherein the aromatic polyamino monomer contains one fluorinated aromatic ring, the fluorinated aromatic ring contains two para-amino groups, and the aromatic polyaldehyde monomer has one of the following structures: 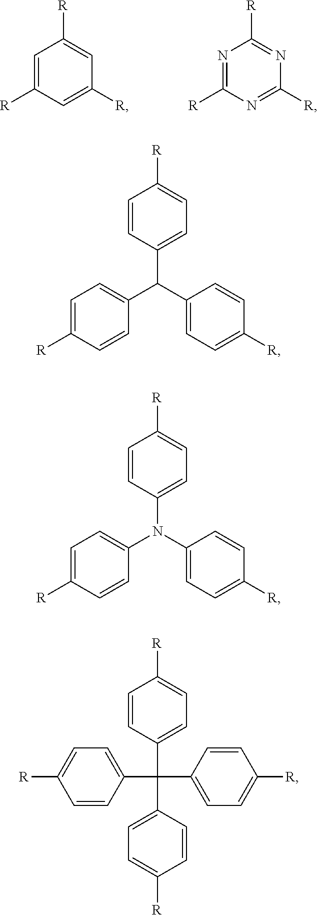
wherein R is aldehyde or 4-aldehyde phenyl.

6. The method according to claim 1, wherein the aromatic polyamino monomer is 1,3,5-tris(4-aminophenyl)benzene or tetra(4-aminophenyl) methane.

7. The method according to claim 1, wherein the aromatic polyaldehyde monomer is 2,5-difluoro-p-phthalaldehyde.
